(19) United States
(12) Patent Application Publication
(10) Pub. No.: US 2022/0059200 A1
(43) Pub. Date: Feb. 24, 2022

(54) DEEP-LEARNING SYSTEMS AND METHODS FOR MEDICAL REPORT GENERATION AND ANOMALY DETECTION

(71) Applicant: Washington University, St. Louis, MO (US)

(72) Inventors: Kasra Rahbar, St. Louis, MO (US); Akash Kansagra, St. Louis, MO (US)

(73) Assignee: Washington University, St. Louis, MO (US)

(21) Appl. No.: 17/409,379

(22) Filed: Aug. 23, 2021

Related U.S. Application Data
(60) Provisional application No. 63/068,587, filed on Aug. 21, 2020.

(51) Int. Cl. G16H 15/00; G16H 50/20; G16H 50/70

(52) U.S. Cl. CPC G16H 15/00; G16H 50/70; G16H 50/20

(57) ABSTRACT

Systems and methods for generating and detecting anomalies in medical reports using an attention-based machine learning model are disclosed. The attention-based machine learning model for generating medical reports includes at least one decoder layer in which each decoder layer includes an attention sublayer operatively coupled to a feed-forward sublayer. The attention-based machine learning model for detecting anomalies in medical reports includes at least one bidirectional encoder layer in which each encoder layer includes an attention sublayer operatively coupled to a feed-forward sublayer.

### CROSS-REFERENCE TO RELATED APPLICATIONS

This application claims priority from U.S. Provisional Application Ser. No. 63/068,587 filed on Aug. 21, 2020, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE DISCLOSURE

The present disclosure generally relates to automated methods of generating medical reports and detecting anomalies in medical reports.

### BACKGROUND

Radiologists diagnose diseases from medical images and communicate their findings to other physicians by means of a report. The report is the final product of radiologists' interpretation and typically contains a detailed description of imaging findings, a differential diagnosis, and recommendations for additional testing or treatment. Using a computational model to understand these reports and generate new reports is a conceptually challenging task because the structure and content in reports can be highly variable.

Radiology reports typically contain a radiologist's interpretation of a medical imaging study. Such reports serve primarily to answer a diagnostic question about a patient's illness but often fulfill multiple additional purposes.

Radiology reports may also provide detailed descriptions of the imaging findings, anatomical considerations that may impact operative planning, and recommendations for additional work-up. As such, unstructured radiology report texts represent rich and complex sources of information about a patient's health.

Current machine learning research in radiology is largely centered on deep learning for computer vision tasks, such as automated detection of pneumonia on chest radiographs. There has been comparatively less emphasis on deep learning based on radiology report text, despite the fact that deep learning networks are particularly suited for complex natural language processing. The suitability of deep learning networks for natural language tasks and the richness of content within radiology reports suggest a significant opportunity for machine learning research in this domain.

Natural language processing involves a language model, which is a statistical model of natural language in the form of a probability distribution over sequences of words. This probability distribution encodes general features of language such as grammatical structure as well as domain-specific knowledge learned from the training data. Taken together, these general and domain-specific features can enable downstream, language-based tasks such as text summarization, machine translation, and question answering.

In language modeling, each token in a text sequence is predicted based on tokens that come before it. This approach is suitable for generating new sequences of text by generating each token sequentially based on the previously generated tokens. This method is unidirectional since it can only look backward at previous tokens for context and cannot look forwards to positions ahead of the current token.

Autoencoding, another type of natural language processing, includes mapping an input sequence to itself using any one or more of at least several approaches. Denoising, one autoencoding strategy, reconstructs an entire original sequence from a corrupted input sequence. Unlike language modeling, in some approaches, autoencoding may make use of bidirectional context, in which context both backward and forwards of the current position is considered. One advantage of bidirectionality is that it makes use of greater contextual information compared to a unidirectional approach.

A bidirectional autoencoder configuration has been used in BERT (“Bidirectional Encoder Representations from Transformers”) for unsupervised pre-training to boost performance on downstream supervised tasks. To date, unsupervised pre-training has been the principal use case for bidirectional autoencoders.

### SUMMARY

Among the various aspects of the present disclosure is the provision of systems and methods for generating and detecting anomalies in medical reports using a machine learning model that incorporates an attention mechanism that implements natural language modeling in terms of the meaning of the word obtained via embedding as well as the positional embedding of each word within the report.

In one aspect, a computer-aided method of automatically generating a medical report is described that includes receiving, using a computing device, a target sequence comprising a plurality of input tokens. The method also includes transforming, using the computing device, the plurality of input tokens of the target sequence into the medical report using a deep learning model. The deep learning model includes at least one decoder layer with an attention sublayer operatively coupled to a feed-forward sublayer. The method further includes displaying the medical report to a clinical practitioner. In some aspects, the medical report comprises at least one section selected from a clinical indication section and an impression section. In some aspects, the type of medical report comprises at least one of a radiology report, a mammography report, a biopsy report, and a colonoscopy report. In some aspects, the at least one decoder layer comprises at least six decoder layers. In some aspects, the attention sublayer of each decoder layer comprises a multi-head self-attention sublayer comprising at least two attention heads. In some aspects, the multi-head self-attention sublayer comprises from about 8 attention heads to about 32 attention heads. In some aspects, the feed-forward sublayer of each decoder layer comprises one of a position-wise fully connected feed-forward network and a sparsely gated mixture-of-experts (MoE) layer. In some aspects, the sparsely gated mixture-of-experts (MoE) layer comprises a gating network operatively coupled to a set of expert networks, wherein the gating network directs an input of the attention sublayer to at least a portion of the set of expert networks, and an output of the MoE layer comprises a weighted sum of at least a portion of expert outputs from the set of expert networks. In some aspects, the set of expert networks comprises at least 2 expert networks. In some aspects, the set of expert networks comprises from about 2 to about 512 expert networks. In some aspects, the weighted sum of at least a portion of the expert network outputs comprises a weighted sum of expert outputs from at least two expert networks with the highest gating values from the gating network. In some aspects, the weighted sum of at least a portion of the expert network outputs further comprises an auxiliary loss term. In some aspects, the method further comprises transforming the target sequence into a plurality of input vectors using an input embedding sublayer operatively coupled to the at least one decoder layer, each input vector comprising an input embedding vector and an associated positional encoding. In some aspects, the method further comprises sampling outputs of the at least one decoder layer using a sampling layer operatively coupled to the at least one decoder layer, the sampling layer configured to transform the outputs of the at least one decoder layer into the medical report. In some aspects, the sampling layer comprises one of a softmax layer, a beam decoding layer, and a nucleus sampling layer. In some aspects, transforming the target sequence into the medical report further comprises sampling the outputs of the at least one decoder layer using random sampling to generate at least one section from the group consisting of the examination summary, the clinical history, and the findings section. In some aspects, the method further comprises selecting a type of medical report to generate based on at least one examination code in the input sequence.

In another aspect, a computer-aided method of generating an impression section for a medical report is disclosed. The method includes receiving, using a computing device, a target sequence that includes a plurality of input tokens. The method also includes transforming, using the computing device, the target sequence into the impression section using the deep learning model described above. The method further includes displaying the medical report to a clinical practitioner.

In an additional aspect, a computer-aided method of detecting anomalies in a medical report is disclosed. The method includes receiving, using a computing device, a target sequence encoding the medical report. The method also includes transforming, using the computing device, at least a portion of the target sequence into a plurality of input tokens, each token comprising an input embedding vector and an associated positional encoding. The method further includes transforming, using the computing device, the plurality of input tokens into an output using a deep learning model. The deep learning model includes at least one bidirectional encoder layer. Each bidirectional encoder layer includes an attention sublayer operatively coupled to a feed-forward sublayer. The method additionally includes sampling, using the computing device, the output to identify each output probability associated with each input token, and classifying, using the computing device, each input token according to an anomaly detection rule. The anomaly detection rule includes classifying each input token as a potential anomaly based on each output probability. The method also additionally includes displaying the medical report to a user, wherein each potential anomaly is indicated to the user. In some aspects, the method further includes sampling, using the computing device, an output probability distribution of each input token classified as a potential anomaly to obtain at least one suggested correction, wherein each suggested correction is selected according to a correction rule; and displaying, using the computing device, the medical report to a user, wherein at least a portion of the at least one suggested correction is displayed with each potential anomaly. In some aspects, the at least one encoder layer comprises at least six encoder layers. In some aspects, wherein the attention sublayer of each encoder layer comprises a multi-head self-attention sublayer comprising at least two attention heads. In some aspects, the multi-head self-attention sublayer comprises from about 8 attention heads to about 32 attention heads. In some aspects, the feed-forward sublayer of each encoder layer comprises one of a position-wise fully connected feed-forward network and a sparsely gated mixture-of-experts (MoE) layer. In some aspects, wherein the sparsely gated mixture-of-experts (MoE) layer comprises a gating network operatively coupled to a set of expert networks, wherein the gating network directs an input of the attention sublayer to at least a portion of the set of expert networks, and an output of the MoE layer comprises a weighted sum of at least a portion of expert outputs from the set of expert networks. In some aspects, the set of expert networks comprises at least 2 expert networks. In some aspects, the weighted sum of at least a portion of the expert network outputs comprises a weighted sum of expert outputs from at least two expert networks with the highest gating values from the gating network. In some aspects, the weighted sum of at least a portion of the expert network outputs further comprises an auxiliary loss term. In some aspects, the method further includes sampling outputs of the at least one encoder layer using a sampling layer operatively coupled to the at least one encoder layer, the sampling layer configured to transform the outputs of the at least one encoder layer into the medical report. In some aspects, the sampling layer comprises one of a softmax layer, a beam decoding layer, and a nucleus sampling layer. In some aspects, the second threshold value corresponds to an associated output probability that is higher than each output probability distribution of each input token. In some aspects, the method further includes training the deep learning model using a training dataset, wherein the training dataset comprises a plurality of ground truth reports and associated training reports, each associated training report comprising the ground truth report with at least one word at a random position altered by substituting the at least one token.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a block diagram schematically illustrating a system in accordance with one aspect of the disclosure.

FIG. 2 is a block diagram schematically illustrating a computing device in accordance with one aspect of the disclosure.

FIG. 3 is a block diagram schematically illustrating a remote or user computing device in accordance with one aspect of the disclosure.

FIG. 4 is a block diagram schematically illustrating a server system in accordance with one aspect of the disclosure.

FIG. 5 is a block diagram illustrating a machine-learning model for generating at least a portion of a medical report in accordance with one aspect of the disclosure.

FIG. 6 is a block diagram illustrating a sparsely gated mixture-of-experts (MoE) layer in accordance with one aspect of the disclosure.

FIG. 7 is a block diagram illustrating a machine-learning model for detecting anomalies in medical reports in accordance with one aspect of the disclosure.

FIG. 8 contains graphs of two hypothetical model distributions for the completion of the sentence “the lungs are” illustrating the difference between accuracy and perplexity.

FIG. 9A is a flow chart describing a first scenario for text generation that includes generating a full-length report for a specified examination type and clinical history.

FIG. 9B is a flow chart describing a second scenario for text generation that includes generating an impression section is generated for a given examination type, clinical history, and preceding report text.

FIG. 10 is an example of a report for a one-view chest radiograph generated using the systems and methods disclosed herein.

FIG. 11 is an example of an impression for a chest CT generated using the systems and methods disclosed herein. The text of the impression section inside of the box is machine-generated from the preceding report text. The model arrives at the diagnosis of tuberculosis based on the characteristic description of imaging findings and clinical history.

### DETAILED DESCRIPTION

In various aspects, a computational method for automated report interpretation and generation is disclosed. The disclosed methods rely on language modeling, as implemented using deep learning techniques. As used herein, a language model refers to a probabilistic model that describes sequences of words that occur in natural language. In various aspects, the language model may be used for understanding natural language and generating new synthetic language samples including, but not limited to, at least portions of medical reports.

In various aspects, the software methodology used to implement the disclosed methods includes a combination of deep learning techniques, architectural components, and additional sources of general medical/radiological knowledge. In some aspects, the architectural components include a deep neural network architecture based on an attention mechanism. Non-limiting examples of suitable attention-based deep neural network architectures include Transformer architectures and Reformer architectures, and variations thereof. In one aspect, the architectural components include a tailored combination of deep neural network architecture and Transformer architecture. Without being limited to any particular theory, the Transformer architecture incorporates an attention mechanism that enables a language model to model dependencies between input and output sequences without regard for their distance in the input or output sequences.

In various other aspects, the deep learning model used to implement the disclosed language model is trained using a broad variety of sources, including, but not limited to, a plurality of reports for medical examinations produced by clinical physicians at one or more clinical institutions or facilities, as well as additional sources of general and/or medical knowledge (e.g., scientific journal articles and online reference articles). The combination of the robust language modeling of the disclosed method and the method's compatibility with a broad range of training data results in a software algorithm that is capable of generating coherent and well-structured medical reports for a wide variety of examination or imaging types, imaging protocols or modalities, and imaging regions.

In various aspects, the disclosed methods may be used in a variety of different applications to automate certain aspects of medical practitioner's workflow. By way of one non-limiting example, the disclosed methods may be used to automatically generate portions or the entirety of a radiology report according to a set of predetermined specifications. By way of a second non-limiting example, the disclosed methods may be used to detect anomalies within existing medical reports including, but not limited to, typographical errors and/or logical inconsistencies, as well as to suggest possible corrections. By way of a third non-limiting example, the disclosed methods may be incorporated into a more general larger interpretive system used to automatically generate a medical report from radiology images or medical images obtained using any other suitable imaging modality.

In various aspects, the architecture of the deep learning model used to implement the disclosed methods of generating and/or detecting anomalies in medical reports incorporate attention layers or attention methods that model global dependencies between input and output sequences independently of separation within the input or output sequences, thereby obviating the need for recurrence or convolution methods and the associated computational resources required. In some aspects, the deep learning models used to implement the disclosed methods may include, but are not limited to, modifications of the existing deep learning models with attention layers.

Non-limiting examples of existing disclosed deep learning models with attention layers suitable for modification to implement the disclosed methods include a Transformer architecture, a Reformer architecture, a BERT architecture, and any other suitable attention-based architecture without limitation. A description of the Transformer architecture is provided in Vaswani et al. (2017), Advances in Neural Information Processing Systems 30: Annual Conference on Neural Information Processing Systems: 6000-6010, incorporated by reference herein in its entirety. A description of the Reformer architecture is provided in Kitaev et al. (2020), https://arxiv.org/pdf/2001.04451.pdf, incorporated by reference herein in its entirety. A description of the BERT architecture is provided in Devlin et al. (2019), https://arxiv.org/pdf/1810.04805.pdf, incorporated by reference herein in its entirety.

Non-limiting examples of suitable language models include XLNet, GPT-2, and any other suitable language model. A description of XLNet is provided in Yang et al. (2019), https://arxiv.org/abs/1906.08237, incorporated by reference herein in its entirety. A description of GPT-2t is provided in Radford et al., https://cdn.openai.com/better-language-models/language_models_are_unsupervised_multitask_learners.pdf, incorporated by reference herein in its entirety.

As described in the examples below, a deep neural network is used to perform unstructured natural language modeling of radiology report text, which contains a wealth of embedded radiological and clinical information. The deep neural network is trained using the Transformer neural network architecture on a dataset of 4.3 million radiology reports from 2010 to 2018 and spanning all body regions and imaging modalities from a large academic radiology practice. Language modeling performance is correlated with increasing model size, particularly with the inclusion of a sparsely gated mixture-of-experts layer that vastly increases model capacity. The disclosed language model learns clinical concepts from contextual clues in reports and synthesizes new report text that mimics human-generated reports. The language model is capable of producing full-length reports that are well structured and clinically plausible. The disclosed deep neural network successfully demonstrates the capability to generate an impression section for existing report text.

As further described in the examples below, an AI-based natural language processing tool is developed to proofread text to identify complex and potentially subtle errors in medical reports. The disclosed tool is based on the Transformer neural network architecture (specifically, as a bidirectional autoencoder) that allows models to learn to detect anomalous tokens (“tokens”=text fragments, such as a word or part of a word) that are judged to be unlikely.

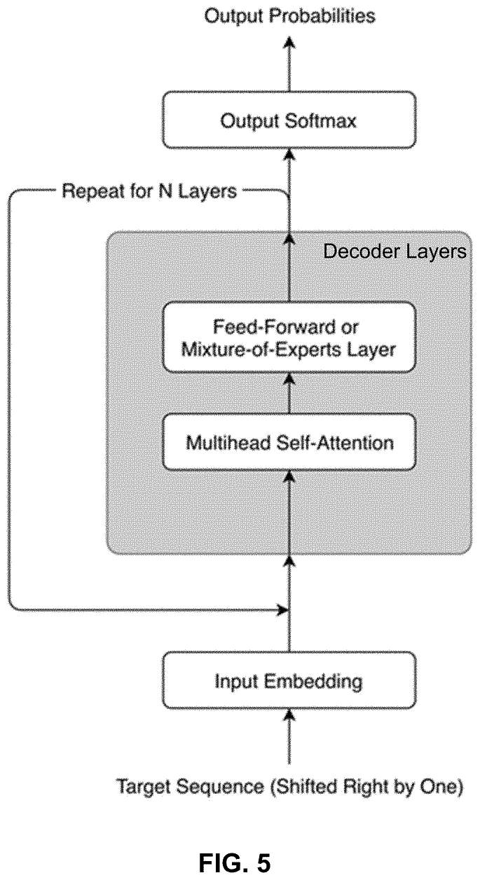

In various aspects, the deep learning model used to implement the method of generating at least a portion of a medical report is illustrated in FIG. 5. As illustrated in FIG. 5, the deep learning model includes at least one decoder layer (shown within the shaded area of FIG. 5). Each decoder layer includes an attention sublayer operatively coupled to a feed-forward sublayer. Without being limited to any particular theory, the number of sublayers may be selected based on the needs of the natural language model selected for use in the disclosed deep learning model. In various aspects, the deep learning model includes one, two, three, four, five, six, 12, 24, 48, or more decoder layers. In some aspects, the deep learning model may adaptively modulate the number of decoder layers as needed.

Referring again to FIG. 5, the attention sublayer operatively connected to the feed-forward sublayer may be any type of attention sublayer implementing any attention method without limitation. In one aspect, the attention sublayer in each decoder layer is a multi-head self-attention sublayer comprising a number of attention heads. Non-limiting examples of suitable multi-head self-attention sublayers are described in Appendix A, as well as in Vaswani et al. (2017). In some aspects, each multi-head self-attention sublayer includes a number of attention heads ranging from about eight attention heads to about 32 attention heads or more.

Referring again to FIG. 5, each decoder layer also includes a feed-forward sublayer. In one aspect, the feed-forward sublayer is a position-wise fully connected feed-forward network similar to the feed-forward sublayer described in Vaswani et al. (2017). In another aspect, the feed-forward sublayer is a sparsely gated mixture-of-experts (MoE) layer. Without being limited to any particular theory, the incorporation of the MoE layer as the feed-forward sublayer of each decoder layer facilitates the inclusion of more trainable parameters without incurring a proportionate increase in required computational resources.

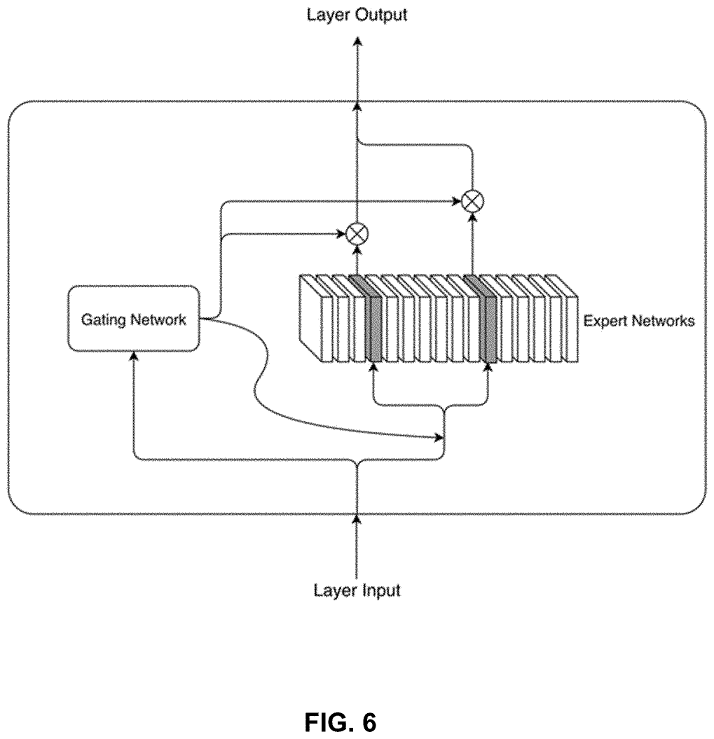

A block diagram illustrating the architecture of a gated MoE layer is provided as FIG. 6. As illustrated in FIG. 6, each MoE layer includes a set of expert networks operatively coupled to a gating network. The gating network directs an input of the attention sublayer to at least a portion of the set of expert networks, and each output of each MoE layer is a weighted sum of at least a portion of expert outputs from the set of expert networks. The subset of expert networks is selected in a data-dependent manner using the gating network that controls which experts are activated for each training example.

In various aspects, the set of expert networks within each MoE layer may be at least 2 expert networks. In various other aspects, the set of expert networks within each MoE layer may be at least 2 expert networks, at least 4 expert networks, at least 8 expert networks, at least 16 expert networks, at least 32 expert networks, at least 64 expert networks, at least 128 expert networks, at least 256 expert networks, and at least 512 expert networks. In one aspect, the set of expert networks within each MoE layer is 32 expert networks.

In one aspect, the output for the entire layer is a weighted sum of the expert outputs in the top K of gating values. In various aspects, the top one expert output, top two expert outputs, top three expert outputs, top four expert outputs, top five expert outputs, top six expert outputs, top seven expert outputs, or top eight expert outputs or more are included in the weighted sum output of the MoE layer. In one aspect, the top two expert outputs are included in the weighted sum output of the MoE layer. In various aspects, an auxiliary loss term is incorporated into the weighted sum output of the MoE layer for load balancing to avoid a few expert networks becoming overly dominant.

Referring again to FIG. 5, at least one decoder layer is operatively coupled to an input embedding layer. In various aspects, the input embedding layer transforms a target sequence, which may include a conditioning sequence and/or one or more parts of a clinician-written summary into one or more input embedding vectors and associated positional encodings. As used herein, the term “input token” may refer to an input embedding vector and associated positional encoding.

In various aspects, the input embedding layer includes a network configured to map the target sequence to a vocabulary. In some aspects, the input embedding layer may be provided as a pre-trained network. In other aspects, the input embedding layer may be trained using a dataset that contains a wide variety of sources of medical and/or scientific terms including, but not limited to, scientific articles, research articles, and any other suitable sources of medical and/or scientific terms.

In various aspects, the input embedding layer may be operatively coupled to any one or more of the at least one encoder layer in any combination without limitation. In one aspect, the output of the input embedding layer may be processed by the first decoder layer, the output of the first decoder layer may be processed by the second decoder layer, and the output of each successive decoder layer may be processed by the next decoder layer, as illustrated in FIG. 5.

In various aspects, the disclosed deep learning model may be sampled using any suitable sampling method without limitation to assemble output probabilities for the plurality of tokens associated with the target sequence. Non-limiting examples of suitable sampling methods include softmax sampling methods, beam decoding methods, nucleus sampling methods, and any other suitable sampling method.

In one aspect, shown illustrated in FIG. 5, the at least one decoder layer may also be coupled to an output softmax layer, which assembles output probabilities for the plurality of tokens associated with the target sequence. In various aspects, a sampling of the output probabilities is used to create the medical report. In one aspect, a predetermined temperature control is used to control the degree of randomness used in the text generation process. In this aspect, a temperature of zero forces the selection of the most probable words (i.e. argmax selection), whereas a temperature of 0.5 implements a random selection of words. In some aspects, the predetermined temperature may be constant throughout the entire process of generating a medical report. In other aspects, different temperatures may be used for generating different sections of the medical report. Without being limited to any particular theory, lower temperatures may be selected to generate portions of the medical report in which precision of language is important, and higher temperatures may be selected within sections in which greater diversity of vocabulary is desired. By way of non-limiting example, the impressions section of a medical report may be generated using a temperature of zero to assure that precise language is maintained in the descriptions of imaging findings, final diagnosis, and recommendations contained in this section. In another non-limiting example, a temperature of 0.5 (corresponding to a random sampling of the output distribution) may be used to produce sections of the generated medical report other than the impressions section, resulting in more vocabulary diversity in sections such as the examination summary, clinical history, and/or findings section.

In various aspects, the methods described above may be used to generate at least a portion of any type of medical report without limitation. Non-limiting examples of medical reports that may be generated using the disclosed deep learning system include radiology reports, histology reports, MRI imaging reports, PET imaging reports, and any other suitable types of medical reports. In some aspects, a full radiology report may include at least one section including, but not limited to, examination codes, an examination summary, a clinical history, a findings section, and an impression section. In other aspects, the methods described above may be used to generate a portion of a medical report including, but not limited to, the impression section.

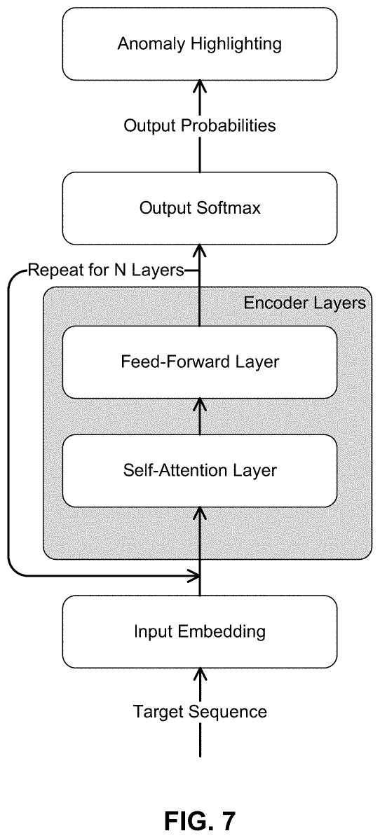

In various aspects, a second deep learning model is used to implement a method of detecting anomalies in a medical report. By way of non-limiting example, a second deep learning model used to implement the method of detecting anomalies in a medical report is illustrated in FIG. 7. The deep learning model includes at least one bi-directional encoder layer (shown within the shaded area of FIG. 7). Each bi-directional encoder layer includes an attention sublayer operatively coupled to a feed-forward sublayer. In various aspects, the deep learning model includes one, two, three, four, five, or six bi-directional encoder layers.

Referring again to FIG. 7, the attention sublayer operatively connected to the feed-forward sublayer may be any type of attention sublayer implementing any attention method without limitation. In one aspect, the attention sublayer in each encoder layer is a multi-head self-attention sublayer comprising a number of attention heads similar to the multi-head self-attention sublayer described above.

Referring again to FIG. 7, each encoder layer also includes a feed-forward sublayer. In one aspect, the feed-forward sublayer is a position-wise fully connected feed-forward network similar to the feed-forward sublayer described above. In another aspect, the feed-forward sublayer is a sparsely gated mixture-of-experts (MoE) layer similar to the MoE layer described above and illustrated in FIG. 6.

Referring again to FIG. 7, at least one encoder layer is operatively coupled to an input embedding layer similar to the input embedding layer described above. In various aspects, the input embedding layer may be operatively coupled to any one or more of the at least one encoder layer in any combination without limitation as described above. In various aspects, the input embedding layer transforms a target sequence, which may include a conditioning sequence and/or one or more parts of a clinician-written summary into one or more input embedding vectors and associated positional encodings. As used herein, the term “input token” may refer to an input embedding vector and associated positional encoding. In some aspects, each input token may correspond to one word within the clinician-written radiology report.

Referring again to FIG. 7, the at least one encoder layer may also be coupled to an output softmax layer, which assembles output probabilities for the plurality of tokens associated with the target sequence. In various aspects, a sampling of the output probabilities is used to detecting anomalies in the medical report.

In various aspects, the output probability associated with each input token is used to detect potential anomalies within the medical report. Referring again to FIG. 7, an anomaly highlighting module operatively coupled to the output softmax layer may be used to evaluate each output probability of each input token to identify errors. In one aspect, each input token is classified according to an anomaly detection rule. In one aspect, the anomaly detection rule includes classifying an input token as a potential anomaly if the associated output probability is less than a threshold value. Any threshold value may be specified by a user without limitation. Without being limited to any particular theory, the threshold value identifies input tokens with output probabilities that are sufficiently low to merit flagging for further review.

In some aspects, no threshold value is used to assess the input tokens and all tokens are indexed with an associated output probability without classification as potential anomalies. In other aspects, a single threshold value may be used for all input tokens of the medical report. In other aspects, the threshold value may vary depending on the position of an input token within the input sequence. By way of non-limiting example, if there is a position within the input sequence for which the probability distribution over possible tokens is very broad, the threshold value may be relatively low, to indicate that many replacement tokens may be potentially appropriate. At another position within the same input sequence, if the probability distribution of the input token is very narrow, then the threshold value may be relatively high to limit the number of replacement tokens that should be considered as potentially appropriate.

In some aspects, the anomaly highlighting module may also identify potential replacement words for those words classified as potential anomalies as described above. In these aspects, the anomaly highlighting module may further sample the output probability distribution to identify replacement tokens that may be selected to replace anomalies. In one aspect, the anomaly highlighting module may sample the output probability distribution of each input token to obtain at least one suggested correction. In this aspect, each suggested correction has an associated output probability that is higher than a second threshold value. Any second threshold value may be selected by a user without limitation, so long as the selections for suggested correction have a higher output probability than the corresponding output probability of the potential anomaly. In one aspect, the second threshold value may be selected to ensure that the suggested correction corresponds to the highest probability from the output probability distribution.

In various aspects, the anomaly detection method described above may further include displaying a highlighted version of the input medical report. In some aspects, the highlighted version is identical to the input medical report, but with each potential anomaly highlighted. In other aspects, each highlighted potential anomaly is further accompanied by a listing of one or more potential corrections identified as described above. In other aspects, all input tokens may be highlighted within the displayed version of the input medical report in proportion to the corresponding output probabilities of the input tokens.

The architecture of the disclosed deep-learning models includes modifications of the existing Transformer architecture for language modeling, which affords at least several advantages over other existing model architectures. Conventional recurrent neural networks become incoherent over long sequences because of the sequential nature of their processing. The Long Short-Term Memory network is a widely used variant that mitigates this issue by using memory to preserve information over long distances. By comparison, Transformer uses a fundamentally different self-attention mechanism to propagate information across long distances, enhancing its ability to generate long sequences of coherent text. As multi-GPU distributed training has become widespread, new parallel training methods such as the MoE layer allow for large models that exceed the memory capacity of a single GPU.

By combining Transformer with the MoE layer, models with as many as 6.6 billion trainable parameters were trained, resulting in improved prediction performance with a larger model size. This observation fits with previous reports that increasing model size improves language modeling performance without over-fitting, even when the number of parameters is extremely large. The benefit of increasing model capacity was most apparent in the diversity of content in the generated text. Larger models are more expressive and generate more interesting findings with descriptions that illustrate a better understanding of concepts.

In some aspects, the language model described herein is generalized by incorporating training data drawn from a variety of institutions to account for varying practices at different institutions. Clinical validation of the generated text output, in particular the generated impressions, will increase confidence in the abilities of the language model, thus encouraging the integration of the systems and methods described above into clinical practice.

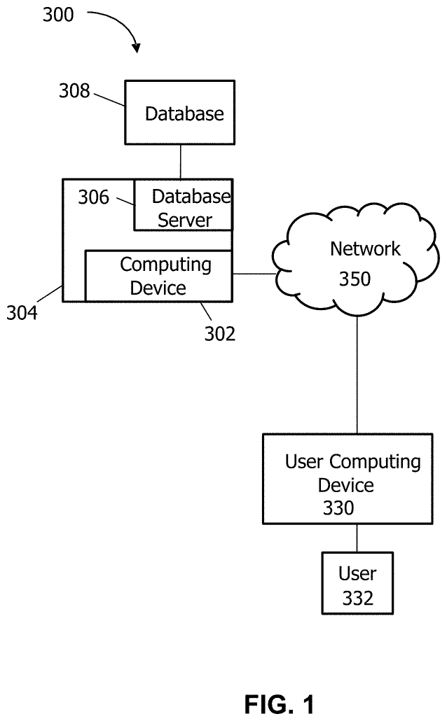

FIG. 1 depicts a simplified block diagram of the system for implementing the computer-aided methods of generating and detecting anomalies in medical reports described herein. As illustrated in FIG. 1, the computing device 300 may be configured to implement at least a portion of the tasks associated with automatically generating or detecting anomalies in a medical report using the attention-based deep learning models described above. The computer system 300 may include a computing device 302. In one aspect, the computing device 302 is part of a server system 304, which also includes a database server 306. The computing device 302 is in communication with a database 308 through the database server 306. The computing device 302 is communicably coupled to a user computing device 330 through a network 350. The network 350 may be any network that allows local area or wide area communication between the devices. For example, the network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user computing device 330 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

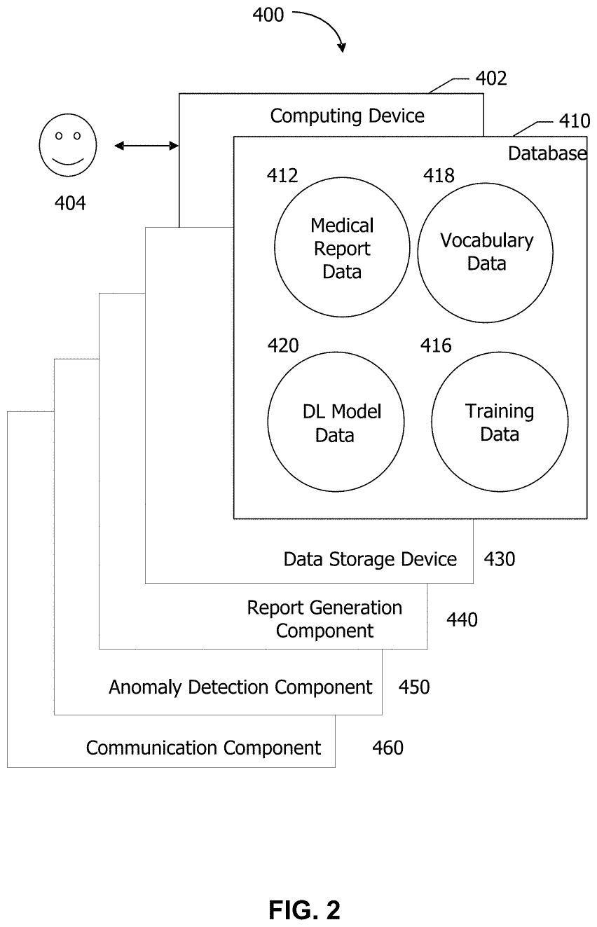

In other aspects, the computing device 302 is configured to perform a plurality of tasks associated with disclosed computer-aided methods of generating and detecting anomalies in medical reports. FIG. 2 depicts a component configuration 400 of computing device 402, which includes database 410 along with other related computing components. In some aspects, computing device 402 is similar to computing device 302 (shown in FIG. 1). A user 404 may access components of computing device 402. In some aspects, database 410 is similar to database 308 (shown in FIG. 1).

In one aspect, database 410 includes vocabulary data 418, DL model data 420, training data 416, and medical report data 412. Non-limiting examples of suitable DL model data 420 include any values of parameters defining the attention-based deep learning models disclosed herein including, but not limited to, parameters defining the at least one encoder layer, the at least one decoder layer, the attention sublayers, the feed-forward sublayers, the input embedding layers, the output softmax layers, the anomaly highlighting layers, and any other parameters defining any other aspect of the attention-based deep learning models, methods of training the attention-based deep learning models, or methods of using the attention-based deep learning models as described herein. In one aspect, the medical report data 412 includes any values defining the target sequence, the clinician-written medical report, the plurality of input tokens, and any other values defining any other data used as input to the attention-based deep learning models as described herein. In one aspect, the vocabulary data 418 includes any values defining the vocabulary used to tokenize the target sequence by the input embedding layer as described herein. In one aspect, training data 416 includes any data used to train the various layers of the attention-based deep learning models as described herein.

Computing device 402 also includes a number of components that perform specific tasks. In an exemplary aspect, computing device 402 includes a data storage device 430, report generation component 440, anomaly detection component 450, and communication component 460. Data storage device 430 is configured to store data received or generated by computing device 402, such as any of the data stored in database 410 or any outputs of processes implemented by any component of computing device 402.

The report generation component 440 enables the generation of a medical report or at least a portion of a medical report such as an impression section using the computer-aided methods as described herein. In various aspects, the report generation component 440 is configured to receive a target sequence, to transform the target sequence into the medical report using a deep learning model.

The anomaly detection component 450 enables anomaly detection within a clinician-written medical report using the computer-aided methods as described herein. In various aspects, the report generation component 440 is configured to receive a target sequence that includes the medical report, to transform at least a portion of the target sequence into a plurality of input tokens, to transform the plurality of input tokens into an output probability distribution using a deep learning model, to sample the output probability distribution to identify each output probability associated with each input token and classifying each input token according to an anomaly detection rule to identify potential anomalies.

Communication component 460 is configured to enable communications between computing device 402 and other devices (e.g. user computing device 330 shown in FIG. 1) over a network, such as a network 350 (shown in FIG. 1), or a plurality of network connections using predefined network protocols such as TCP/IP (Transmission Control Protocol/Internet Protocol).

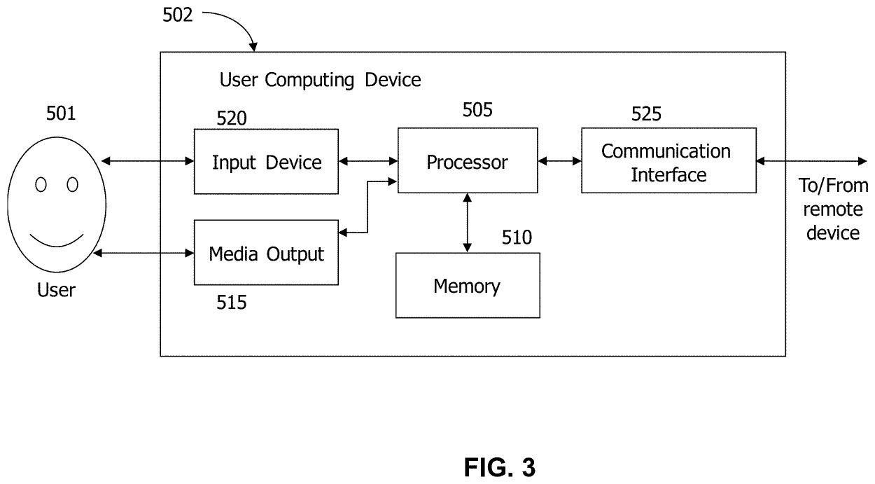

FIG. 3 depicts a configuration of a remote or user computing device 502, such as user computing device 330 (shown in FIG. 1). Computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light-emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WI MAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

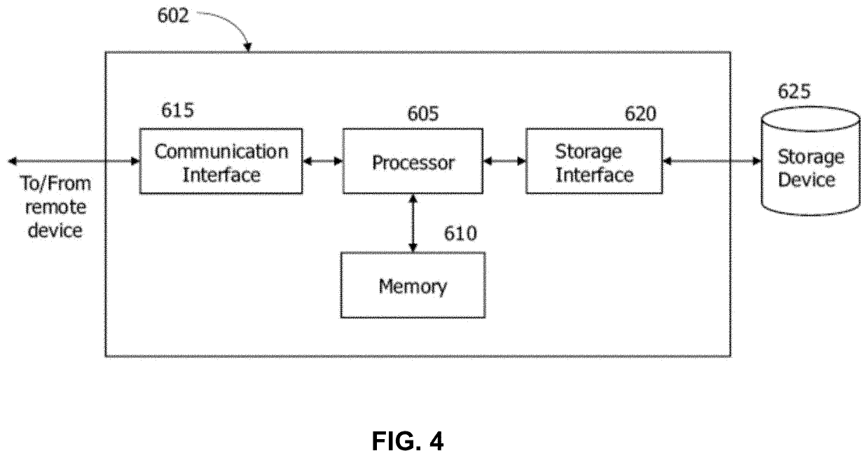

FIG. 4 illustrates an example configuration of a server system 602. Server system 602 may include, but is not limited to, database server 306 and computing device 302 (both shown in FIG. 1). In some aspects, server system 602 is similar to server system 304 (shown in FIG. 1). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 330 (shown in FIG. 1) or another server system 602. For example, communication interface 615 may receive requests from user computing device 330 via a network 350 (shown in FIG. 1).

Processor 605 may also be operatively coupled to a storage device 625. Storage device 625 may be any computer-operated hardware suitable for storing and/or retrieving data. In some aspects, storage device 625 may be integrated into server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIG. 3) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only, and are thus not limiting as to the types of memory usable for the storage of a computer program.

The computer systems and computer-aided methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer-executable instructions stored on non-transitory computer-readable media or medium.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may include but are not limited to images or frames of a video, object characteristics, and object categorizations. Data inputs may further include sensor data, image data, video data, telematics data, authentication data, authorization data, security data, mobile device data, geolocation information, transaction data, personal identification data, financial data, usage data, weather pattern data, “big data” sets, and/or user preference data. ML outputs may include but are not limited to: a tracked shape output, categorization of an object, categorization of a region within a medical image (segmentation), categorization of a type of motion, a diagnosis based on the motion of an object, motion analysis of an object, and trained model parameters ML outputs may further include: speech recognition, image or video recognition, medical diagnoses, statistical or financial models, autonomous vehicle decision-making models, robotics behavior modeling, fraud detection analysis, user recommendations and personalization, game AI, skill acquisition, targeted marketing, big data visualization, weather forecasting, and/or information extracted about a computer device, a user, a home, a vehicle, or a party of a transaction. In some aspects, data inputs may include certain ML outputs.

In some aspects, at least one of a plurality of ML methods and algorithms may be applied, which may include but are not limited to: genetic algorithms, linear or logistic regressions, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, adversarial learning, and reinforcement learning.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and backup drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

To demonstrate and validate the training and use of an attention-based deep learning model for generating medical reports as described above, the following experiments were conducted.

A large deep learning network to perform language modeling using a very large database of radiology was implemented as described below. The language model of the deep learning network was assessed as described below to demonstrate the fluency of the language model by generating an impression section, which is likely to be a practical and time-saving tool in routine radiology practice.

A dataset of 4.3 million radiology reports for examinations interpreted at a single, large academic practice between 2010 and 2018 was created for use in these experiments. This dataset spanned all body regions and imaging modalities, including radiography (53%), CT (16%), MRI (9%), ultrasound (7%), mammography (7%), interventional procedures (4%), nuclear medicine (3%), and fluoroscopy (1%).

Report text was tokenized using a vocabulary of approximately 32,000 sub-words and converted to lower case to reduce redundancy in the vocabulary. Each report was prepended with a conditioning sequence of input text consisting of the accompanying examination code(s) and a clinical history. In some cases, this conditioning sequence can be used to control the type of report generated by the deep learning network.

### Model Description

The deep learning model was a modification of an existing Transformer architecture. For these experiments, only the decoder block was used and the encoder-decoder attention layers were omitted, as illustrated in FIG. 5. The original feed-forward layer of the existing Transformer architecture (FIG. 5) was substituted with a sparsely gated mixture-of-experts (MoE) layer to increase the number of trainable parameters (FIG. 6). As illustrated in FIG. 6, the MoE layer included a set of expert networks, a subset of which were selected in a data-dependent manner using a gating network that controlled which experts were activated for each training example. The output for the entire layer was a weighted sum of the expert outputs in the top K of gating values (K=2 in the implementation in these experiments). An auxiliary loss term was used for load balancing to avoid a few experts becoming overly dominant.

Models of different sizes were tested by adjusting model dimension, the number of attention heads, and the hidden dimension of the original feed-forward or MoE layers. The total number of layers for all tested models was six and the number of experts for each MoE layer was 32. Models were implemented using the tensor2tensor library. The Adafactor optimizer was used along with mixed-precision training.

### Performance Assessment

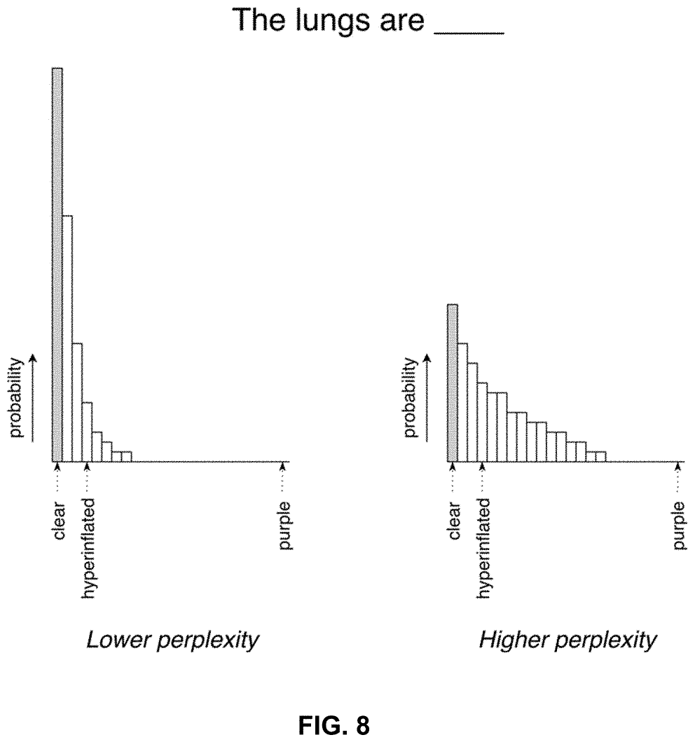

The model described above made predictions for each position in a text sequence by generating a probability distribution across words in the vocabulary. The performance of the language model can be quantitatively measured from this probability distribution with widely used metrics including, but not limited to, accuracy and perplexity. Accuracy measured the concordance between the single highest probability word and the ground-truth word, regardless of predicted probability, across all positions in the text. Perplexity accounted for the probability placed on the ground-truth word across all positions in the text. FIG. 8 contains two histograms showing hypothetical model distributions for the completion of the sentence “the lungs are ______”. The words in the distributions are ordered by decreasing probability, where “clear” is the highest probability word, “hyperinflated” is of intermediate probability, and “purple” has zero probability. The lower perplexity (left) and higher perplexity (right) model distributions were equal in terms of accuracy, since accuracy only measured the concordance of the highest probability word with the ground-truth word (shaded). However, the model distribution on the right had higher (worse) perplexity because it placed lower probability on the ground-truth word. Higher accuracy and lower perplexity were indicative of better prediction performance.

### Text Generation

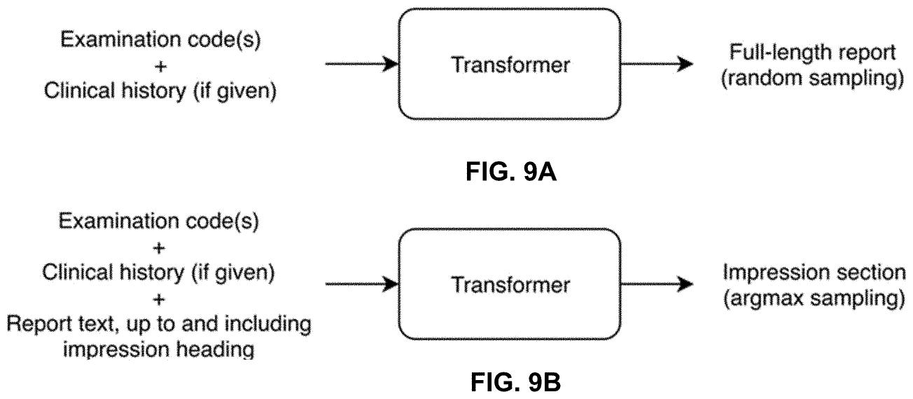

The generated text samples presented here were from the largest model listed in Table 1 below with 6.6 billion parameters. Text generation is performed for two scenarios, as summarized in FIGS. 9A and 9B.

TABLE 1
Language model size and performance for
different model hyperparameters

| Model hyperparameters | | | | | |
|---|---|---|---|---|---|
| | | Size of | Model size | Model performance | |
| | | feed- | Number of | Perplexity | Accuracy |
| Model | Attention | forward or | parameters | (lower is | (higher is |
| dimension | heads | MoE layers | (in millions) | better) | better) |
| Transformer | | | | | |
| 512 | 8 | 2048 | 35 | 2.83 | 74.3 |
| 1024 | 16 | 16384 | 638 | 2.23 | 79.0 |
| Transformer with MoE layers | | | | | |
| 1024 | 16 | 8192 | 2827 | 2.12 | 80.3 |
| 1024 | 32 | 16384 | 6577 | 2.13 | 80.5 |

Bold numbers indicate best performance.

The results from the more open-ended first scenario, illustrated in FIG. 9A, demonstrated the expressiveness and clinical fluency of the language model by generating a full-length report from an input consisting of examination code(s) and clinical history if provided. The report text was generated by sampling the model distribution using random sampling with a softmax temperature value of 0.5. Temperature controlled the degree of randomness in text generation, with a temperature of zero forcing the selection of the most probable word (which is equivalent to argmax sampling) and a higher temperature permitting random sampling of words according to their probability, thereby allowing for greater diversity in the generated text. A temperature value of 0 forces selection of the most probable word at each position in the sequence; higher temperature permits sampling of words other than the most probable word and thereby allows for greater diversity in the generated text.

The results of the second scenario, illustrated in FIG. 9B, demonstrated a complex but practical application of text generation capability, by generating the “impression” section that typically concludes essentially all radiology reports. The impression section summarized and prioritized imaging findings, provided a final diagnosis that took into account imaging findings as well as patient history, and recommended further management steps when appropriate. Because generating the impression section was more close-ended than the first scenario of generating a full-length report, text generation was performed using argmax sampling (picking the most probable word at each position in the sequence). The deep-learning model in the second scenario used the report text preceding the impression to generate the as-yet-incomplete impression with a sampling temperature value of 0, as a diversity of generated text is not desirable in this scenario.

### Results

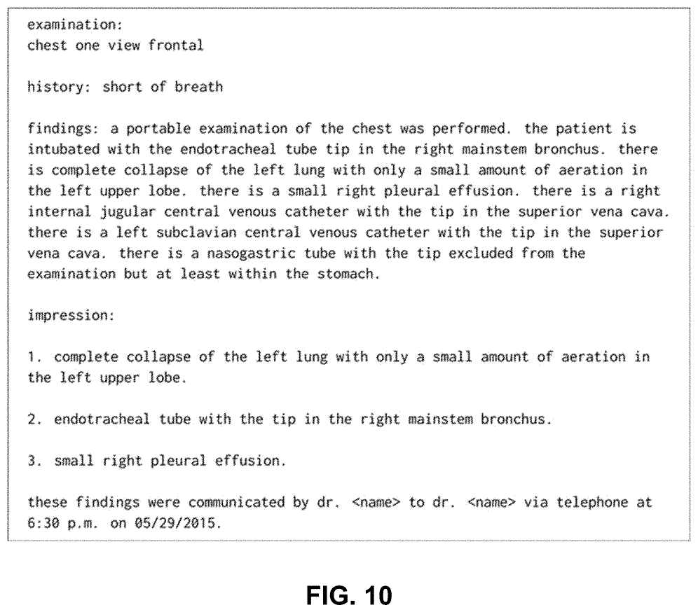

FIG. 10 is a sample report for a portable chest radiograph generated using the deep-learning model described above under scenario 1 (FIG. 9A). The qualitative results demonstrated impressive fluency of the language model. For example, the report shown in FIG. 10 stated that an endotracheal tube was in the right mainstem bronchus followed by a contextually plausible description of left lung collapse. The report also demonstrated consistency across sections, with the impression section accurately summarizing and prioritizing the findings in the form of an enumerated list that places the clinically important findings of lung collapse and endobronchial intubation ahead of the less significant finding of a small pleural effusion. Finally, the reports generated in this experiment provided expected documentation of communication with the referring physician in the presence of a clinically actionable finding.

Additional reports (not illustrated herein) were generated to demonstrate the generalizability of the language model. In a generated report for a pediatric elbow radiograph, the possibility of an occult fracture in the presence of a joint effusion yielded an appropriate recommendation for radiographic follow-up. In a generated report for a CT of the abdomen and pelvis, appendicitis was a contextually appropriate cause for right lower quadrant pain in a young adult and the report text also commented on the pertinent absence of perforation or abscess. In a generated report combining radiographs of multiple body parts, the report was appropriately structured and internally consistent across different sections with separate descriptions of findings and impression points for each body part.

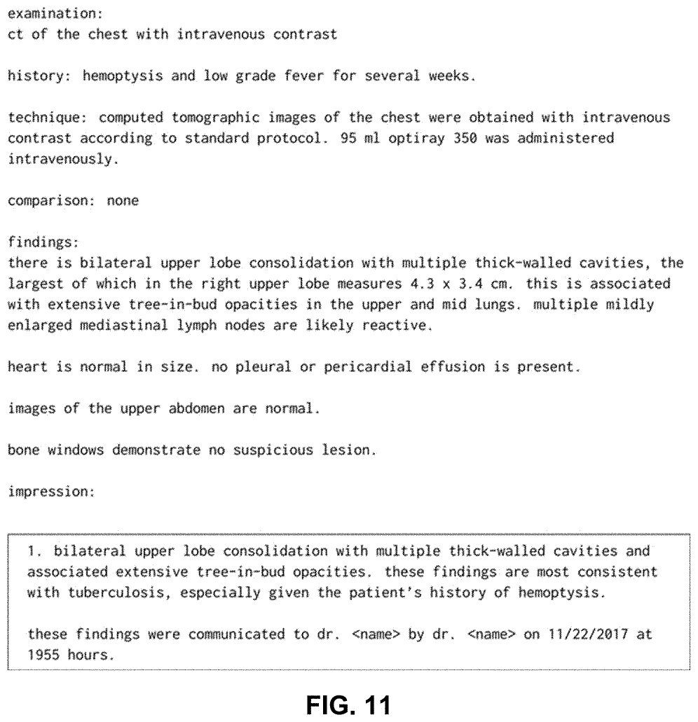

FIG. 11 is a sample impression for a chest CT report in which the provided clinical history and imaging findings were suggestive of tuberculosis, though the diagnosis is not named in the CT report. The qualitative results demonstrated thoughtful and clinically reasonable impressions that were consistent with the remainder of the report. The deep-learning model correctly synthesized the supporting evidence in the clinical history and imaging findings to arrive at the correct diagnosis.

Additional impressions (not illustrated herein) were generated to demonstrate the generalizability of the language model. In a generated impression for a knee radiograph, the deep lateral femoral notch sign, which was synonymous with the deep sulcus sign, was noted in the findings, and the impression identified the association with an anterior cruciate ligament tear. In a generated impression for a brain MRI, the model correctly translated a characteristic description of a suprasellar mass into the correct diagnosis of craniopharyngioma, and additional unrelated findings were appropriately summarized in separate impression points. Three impressions were generated for a head CT in the setting of suspected ischemic stroke. Adjusting the saliency and severity of the findings of cerebral edema (highlighted) appropriately altered the expressed confidence in the diagnosis of ischemic stroke. Three additional impressions (not illustrated herein) were produced for a chest CT with an incidental pulmonary nodule. Adjusting the description of nodule size and morphologic features appropriately altered the suspicion for malignancy and recommended imaging follow-up.

Quantitative results of performance assessment in the form of model perplexity and accuracy are reported in Table 1 above. Prediction performance was positively correlated with model size, with the best-performing models containing billions of trainable parameters. However, there was only a modest benefit to increasing the model size from 2.8 billion to 6.6 billion parameters.

### Discussion

The implementation of a language model trained on 4.3 million radiology reports spanning the entire spectrum of modern radiological practice was described above. The fluency of this language model was demonstrated by generating clinically sensible full-length reports. Automatical generation of the impression section based on preceding report content was also demonstrated. The quantitative measures of model performance and the examples of generated reports were comparable to what a radiologist might produce in actual practice.

The results of these experiments represent proof of concept that the unique deep learning architecture described above is well-suited for use as a platform for language task development. Writing the impression section is a particularly time-consuming and cognitively demanding aspect of composing most radiology reports, and automating this tedious task may allow radiologists to direct greater focus to other tasks such as image interpretation.

In conclusion, deep learning networks and a modified Transformer architecture can be used to implement a powerful natural language model, particularly with large model sizes. This language model can be used to generate fluent, clinically meaningful text that can be used to automate the composition of the impression section, a task that may be of significant practical value in current radiology practice.

### Example 2

To demonstrate and validate the training and use of an attention-based deep learning model for anomaly detection within medical reports as described above, the following experiments were conducted.

### Language Modeling Versus Autoencoding

In language modeling, each token in a text sequence is predicted based on tokens that come before it. This approach is suitable for generating new sequences of text by generating each token sequentially based on the previously generated tokens. This method is unidirectional since it can only look backward at previous tokens for context and cannot look forwards to positions ahead of the current token.

In autoencoding, the aim is to reconstruct the entire original sequence from a corrupted input sequence. Unlike language modeling, autoencoding is bidirectional, making use of context both backward and forwards of the current position. The advantage of bidirectionality is that it makes use of greater contextual information compared to the unidirectional approach.

A bidirectional autoencoder configuration has been used in BERT (“Bidirectional Encoder Representations from Transformers”) for unsupervised pre-training to boost performance on downstream supervised tasks. Thus, a bidirectional autoencoder has only been used as an adjunctive tool to improve training quality, not to generate output from the trained model. A bidirectional autoencoder was developed and implemented that was directly applied to a proofreading task. This approach was enabled by utilizing model-derived probabilities of tokens at each position in a text sequence and conceptually equating low probability tokens to probable errors.

In a denoising autoencoder, a corrupted input sequence was evaluated and the task of the model was to recover the original sequence. The method of data corruption of the input sequence was crucial in order to perform the proofreading task. The noising method used in BERT was to replace random tokens in the input sequence with a MASK token. While simple and adequate for unsupervised pre-training, this approach was insufficient for the proofreading task described herein. The model should not know in advance which tokens were going to be errors; simply masking random tokens removed the challenge of identifying the location of the errors for the model. The model needed the dual challenge of both determining the location of the errors and recovering the original input. To this end, a strategy of substituting tokens at random positions from the vocabulary (with probability proportional to frequency rank) was developed to allow training suitable for the proofreading task.

What is claimed is:

1. A computer-aided method of automatically generating a medical report, the method comprising:
a. receiving, using a computing device, a target sequence comprising a plurality of input tokens;
b. transforming, using the computing device, the plurality of input tokens of the target sequence into the medical report using a deep learning model, the deep learning model comprising at least one decoder layer, each decoder layer comprising an attention sublayer operatively coupled to a feed-forward sublayer; and
c. displaying, using the computing device, the medical report to a clinical practitioner.

2. The method of claim 1, wherein the type of medical report comprises at least one of a radiology report, a mammography report, a biopsy report, and a colonoscopy report.

3. The method of claim 1, wherein the medical report comprises at least one section selected from an examination summary, a clinical indication section, a clinical history, a findings section, and an impression section.

4. The method of claim 1, wherein the at least one decoder layer comprises at least six decoder layers.

5. The method of claim 1, wherein the attention sublayer of each decoder layer comprises a multi-head self-attention sublayer comprising at least two attention heads.

6. The method of claim 5, wherein the multi-head self-attention sublayer comprises from about 8 attention heads to about 32 attention heads.

7. The method of claim 1, wherein the feed-forward sublayer of each decoder layer comprises one of a position-wise fully connected feed-forward network and a sparsely gated mixture-of-experts (MoE) layer.

8. The method of claim 7, wherein the sparsely gated mixture-of-experts (MoE) layer comprises a gating network operatively coupled to a set of expert networks, wherein the gating network directs an input of the attention sublayer to at least a portion of the set of expert networks, and an output of the MoE layer comprises a weighted sum of at least a portion of expert outputs from the set of expert networks, the weighted sum of at least a portion of the expert network outputs comprising a weighted sum of expert outputs from at least two expert networks with the highest gating values from the gating network.

9. The method of claim 8, wherein the set of expert networks comprises from about 2 to about 512 expert networks.

10. The method of claim 9, wherein the weighted sum of at least a portion of the expert network outputs further comprises an auxiliary loss term.

11. The method of claim 1, further comprising transforming, using the computing device, the target sequence into a plurality of input vectors using an input embedding sublayer operatively coupled to the at least one decoder layer, each input vector comprising an input embedding vector and an associated positional encoding.

12. The method of claim 1, further comprising sampling, using the computing device, outputs of the at least one decoder layer using a sampling layer operatively coupled to the at least one decoder layer, the sampling layer configured to transform the outputs of the at least one decoder layer into the medical report.

13. The method of claim 12, wherein the sampling layer comprises one of a softmax layer, a beam decoding layer, and a nucleus sampling layer.

14. The method of claim 1, wherein transforming the target sequence into the medical report further comprises sampling, using the computing device, the outputs of the at least one decoder layer to generate at least a portion of the medical report using at least one of argmax sampling and random sampling.

15. The method of claim 1, further comprising selecting, using the computing device, a type of medical report to generate based on at least one examination code in the input sequence.

16. The method of claim 1, wherein transforming the plurality of input tokens of the target sequence into the medical report using a deep learning model further comprises transforming, using the computing device, the target sequence into the impression section and appending, using the computing device, the impression section to the target sequence to generate the medical report.

17. A computer-aided method of detecting anomalies in a medical report, the method comprising:
a. receiving, using a computing device, a target sequence encoding the medical report;
b. transforming, using the computing device, at least a portion of the target sequence into a plurality of input tokens, each input token comprising an input embedding vector and an associated positional encoding;
c. transforming, using the computing device, the target sequence into an output using a deep learning model, the deep learning model comprising at least one bidirectional encoder layer, each bidirectional encoder layer comprising an attention sublayer operatively coupled to a feed-forward sublayer;
d. sampling, using the computing device, the output to identify each output probability associated with each input token;
e. classifying, using the computing device, each input token according to an anomaly detection rule, the anomaly detection rule comprising classifying each input token as a potential anomaly if the associated output probability is less than a threshold value; and
f. displaying, using the computing device, the medical report to a user, wherein each potential anomaly is indicated to the user.

18. The method of claim 17, further comprising sampling, using the computing device, an output probability distribution of each input token classified as a potential anomaly to obtain at least one suggested correction, wherein each suggested correction is selected according to a correction rule.

19. The method of claim 18 wherein displaying the medical report to the user further comprises displaying at least a portion of the at least one suggested correction with each potential anomaly.
